(19) United States
(12) Patent Application Publication
(10) Pub. No.: US 2005/0136142 A1
(43) Pub. Date: Jun. 23, 2005

(54) Extract from the roots or stems of urticaceae for hepatitis B therapy

(75) Inventors: Lain-Tze Lee, Hsinchu (TW); Shau-Feng Chang, Hsinchu (TW); Cheng-Yu Lee, Hsinchu (TW); Shu-Jiau Chiou, Hsinchu (TW); Tien-Soung Tong, Hsinchu (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsinchu (TW)

(21) Appl. No.: 10/915,465

(22) Filed: Aug. 11, 2004

(30) Foreign Application Priority Data
Dec. 19, 2003 (TW) 092136272

(51) Int. Cl. A61K 35/78; A61K 48/00

(52) U.S. Cl. 424/773; 424/779; 514/44; 514/12

(57) ABSTRACT

An extract for treating hepatitis extracted from the roots or stems of Urticaceae by organic solvents is disclosed. The Urticaceae used in the present invention has a unique sequence of internal transcribed spacer of rDNA, which is at least 70% sequence similarity with that of Boehmeria frutescens, Boehmeria zollingeriana, Urlica thunbergiana, Pilea spp., Boehmeria nivea or Boehmeria densiflora. The extract of the present invention can inhibit the DNA replication of both the wild type hepatitis B virus and the lamivudine-resistant hepatitis B virus.

### BACKGROUND OF THE INVENTION

1. Field of the Invention

The present invention relates to a medicinal plant extract. The extract inhibits the viral DNA replication as well as secretion of the two antigens, surface antigen (HBs) and e antigen in hepatitis B virus transformed cell cultured system. Furthermore, inhibition activities of viral replication and secretion proteins have been found not only in the wild type HBV but also in the lamivudine-resistant HBV. The medicinal plants from Urticaceae is used for treating hepatitis that have a specific ribosomal DNA internal transcribed spacer (ITS), moreover ITS is used to identify the consensus of said medicinal plant.

2. Description of Related Art

There are 350 million people worldwide suffering from chronic Hepatitis B carriers. In the US alone, about 1.25 million people were infected with chronic hepatitis. As for China, about 30 to 50 million people have chronic hepatitis B infection, and 120 million people are chronic hepatitis B carriers. Among them, about 300 thousand people die from hepatitis B annually. In Taiwan's case, about 3 million people are infected with HBV.

In the US, it is estimated that about 140-320 thousand people are infected with HBV every year. The cost to the national economy due to HBV infection is at least 700 million US dollars per year. In Taiwan, the cause of death from chronic disease and cirrhosis are calculated to be more than 5000 people, and it has become the sixth commonest cause of death in this country. That figure would be considerably more when tumor-associated liver diseases are also included in the above statistic. is.

Presently, interferon and lamivudine are the major therapeutically drugs for hepatitis B infection in clinical. In 1992, interferon was approved by the FDA for treating hepatitis B. However, serious side effects occur during the treatment, and there are only 20% of hepatitis B patients who respond satisfactorily to interferon. In 1998, lamivudine was approved by the FDA for treating hepatitis B, but only 17-33% of treated patients responded well. Lamivudine also did not responded well in the Chinese market. What is even worst about lamivudine is it can cause mutation of the hepatitis B virus after long-term usage of lamivudine. As usage time prolong the risk of mutation due to lamivudine also increases. Clinical study showed that there is about 24% mutation rate from treatments with lamivudine in the first year, and that increases to 42% in the second year, 52% in the third year, and up to 67% in the fourth year. Therefore, it is necessary to develop a new drug with higher efficacy, lower side effects, and lower virus mutation rate for hepatitis B therapy.

The clinical studies indicated that HBeAg positive patients are associated with higher risk to be infected from hepatocellular carcinoma. The relative risk of hepatocellular carcinoma was 9.6 fold among men who were positive for HBsAg alone and 60.2 fold among those who were positive for both HBsAg and HBeAg, as compared with men who were negative for both (N Engl J Med 2002; 347:168-74.). Therefore, it is vital to develop new drug with high efficacy, prevention of virus mutation, cured drug induced mutant HBV and even new mechanism of inhibition of viral replication for the hepatitis B therapy.

Accordingly, the present invention provides a medicinal plant extract. The extract inhibits the viral DNA replication and secretion of the two antigens, surface antigen and e antigen in HBV transformed cell cultured system. Further, a process using sequence comparison of ribosomal DNA (rDNA) internal transcribed spacer (ITS) is developed to identify said medicinal plant.

### SUMMARY OF THE INVENTION

The objective of the present invention is to provide a medicinal plant extract for treating hepatitis B, which comprises pestled roots or stems of Boehmeria frutescens, Boehmeria zollingeriana, Urlica thunbergiana, Pilea spp., Boehmeria nivea or Boehmeria densiflora.

Another objective of the present invention is to provide extraction methods for preparing the extract to inhibit the viral replication of HBV and secretion of the two antigens, surface antigen and e antigen. The extraction methods comprise pestling the roots or stems of Boehmeria frutescens, Boehmeria zollingeriana, Urlica thunbergiana, Pilea spp., Boehmeria nivea or Boehmeria densiflora to powder, and extracting the powder with solvent to obtain the extract.

The objective of the present invention is further to provide a process of DNA technology to identify a medicinal plant used in treating hepatitis B. The medicinal plant has a specific internal transcribed spacer (ITS) sequence of ribosomal DNA in which at least 70% consensus sequences among that of Boehmeria frutescens, Boehmeria zollingeriana, Urlica thunbergiana, Pilea spp., Boehmeria nivea or Boehmeria densiflora, so as to isolate a medicinal plant preferably inhibiting the DNA replication and the surface antigen and e antigen secretion of HBV.

In one specific embodiment, the present invention applies the medicinal plant extract for treating hepatitis B via virus inhibition assay in vitro: (1) inhibition of the DNA replication as well as secretion of both surface antigen (HBs) and e antigen (HBe) in the wild type of HBV; (2) inhibition of the DNA replication as well as secretion of both surface antigen (HBs) and e antigen (HBe) in the lamivudine-resistant of HBV.

In another specific embodiment, the present invention applies established sequences of the ITS of Boehmeria frutescens, Boehmeria zollingeriana, Urlica thunbergiana, Pilea spp., Boehmeria nivea and Boehmeria densiflora for identifying the said medicinal plant.

Other objects, advantages, and novel features of the invention will become more apparent from the following detailed description when taken in conjunction with the accompanying drawings.

### BRIEF DESCRIPTION OF THE DRAWINGS

FIG. 1 shows the inhibition effect of the Urticaceae extract against the two antigens, surface antigen and e antigen of HBV in transformed human hepatoma cells.

FIG. 2 shows the inhibition effects of the plant extract against two antigens, surface antigen and e antigen of the wild type and the mutated lamivudine-resistant of HBV in transient transfected human hepatoma cells.

FIG. 3 shows the inhibition effect of the plant extract against the DNA replication of the wild type and the mutated lamivudine-resistant of HBV in transformed human hepatoma cells.

FIG. 4 is the ribosomal ITS consensus result of the Urticaceae.

### DETAILED DESCRIPTION OF THE PREFERRED EMBODIMENT

In the present invention, the Urticaceae comprises Boehmeria frutescens, Boehmeria zollingeriana, Urlica thunbergiana, Pilea spp., Boehmeria nivea and Boehmeria densiflora.

The present invention comprises a medicinal plant extract for treating hepatitis B, which is preferably prepared by pestling the roots or stems of Boehmeria frutescens, Boehmeria zollingeriana, Urlica thunbergiana, pilea spp., Boehmeria nivea or Boehmeria densiflora to powder, and then extracting with solvent.

The above preparation is a known process in the art and without specific description, and is included in the scope of the present invention.

The above-mentioned solvent extraction is the solvent extraction of the effective components in the medicinal plant. Thus, a high polar solvent, such as water, methanol, ethanol, or the mixture thereof, but not limited herein, is used for the extraction. The thus obtained extract or the extracted portion is further extracted with low polar solvent, and the defined dielectric constant of the low polar solvent is lower than 10 in the present invention, such as ethyl acetate, difluoromethane, trifluoromethane, tetrafluoromethane, cyclohexane, n-hexane, n-butylalcohol ether, benzene, or the mixture thereof, but not limited herein, for the extraction.

In the embodiment of the present invention, the medicinal plant is first placed in a high polar solvent such as ethanol and refluxed under heat, then a low polar solvent such as ethyl acetate or the proportional water and ethanol solution mixture is added to extract the effective components of the medicinal plant.

After the medicinal plant is pestled, a pre-extraction process comprising methanol, ethanol, or the mixture thereof is used if necessary. As mentioned above, though methanol and ethanol are hydrophilic and are high polar solvents (with the dielectric constant of about 26 to 31), a certain level in methanol or ethanol solubility of the plant cell portions, except for those proteins, oils and waxes, occurs. Therefore, the extraction by using methanol, ethanol, or the mixture thereof is contributive to the following low polar solvent extraction in the present invention.

Another aspect of the present invention is to provide a process of the medicinal plant extraction for treating hepatitis B, which comprises pestling the roots or stems of Boehmeria frutescens, Boehmeria zollingeriana, Urlica thunbergiana, Pilea spp., Boehmeria nivea and Boehmeria densiflora, and then extracting with high polar and low polar solvents. If necessary, a pre-extraction process comprising methanol, ethanol, or the mixture thereof is used before the high polar solvent extraction, and it may contribute to the following low polar solvent extraction of the present invention.

However, to improve the purity of the effective components in the extract, if necessary, several purification processes are performed after the present extraction. The purification process of the extract is used without specific description, and is a known skill in the art. The preferred processes include, for example, chromatography, crystallography, filtration, precipitation and so on, and are determined with desired goals.

Another medicinal plant extract for treating hepatitis B in the present invention is to pestle the roots or stems of Boehmeria frutescens, Boehmeria zollingeriana, Urlica thunbergiana, Pilea spp., Boehmeria nivea or Boehmeria densiflora, and then extracts with solvents, to produce an extract which can inhibit both wild type and lamivudine-resistant HBV.

The above mentioned inhibition effect of the medicinal plant extract against both surface antigen and e antigen in the wild type as well as the lamivudine-resistant of HBV is demonstrated in tested cell cultured systems.

In one specific embodiment, the present invention applies the medicinal plant extract for treating hepatitis B to inhibit virus in vitro: (1) inhibition of the viral DNA replication and the secretion of both surface antigen and e antigen in the wild type of HBV; (2) inhibition of the viral DNA replication and the secretion of both surface antigen and e antigen in the lamivudine-resistant of HBV.

The present medicinal plant extract for treating hepatitis B may be combined with other substances, for example, other antiviral drugs or the mixture thereof, or nutrients, to enhance the combination in antiviral effect.

The present extract can be alone or combined with the pharmaceutical acceptable carrier or excipient, and administrated in the form of single or multiple dosages. A pharmaceutically acceptable carrier or diluent, and any other known adjuvant and excipient can be prepared in accordance with the conventional skills, for example, see Remington's Pharmaceutical Sciences, version 19^{th}, Gennaro editorial, Mack Pub. Co., Easton, Pa. (1995).

A further aspect of the present invention is to provide a method to identify a medicinal plant that is also used for treating hepatitis B. The ITS sequence from rDNA of an unidentified Urticaceae is compared to that of Boehmeria frutescens, Boehmeria zollingeriana, Urlica thunbergiana, Pilea spp., Boehmeria nivea or Boehmeria densiflora in which the sequence similarity should not be higher than 70%.

The above mentioned method to identify a medicinal plant for treating hepatitis B comprises the following steps: extract the total DNAs of Urticaceae, amplify the ITS segment of rDNA from total extracted DNA by using polymerase chain reaction (PCR), and obtain the ITS sequences of Urticaceae by direct sequecing. Consequently, the database of ITS sequences of Urticacea comprising Boehmeria frutescens, Boehmeria zollingeriana, Urlica thunbergiana, Pilea spp., Boehmeria nivea and Boehmeria densiflora is established, which can be applied to identify a medicinal plant for treating hepatitis B.

The above mentioned method to identify a medicinal plant for treating hepatitis B explores the ITS sequences of Boehmeria frutescens, Boehmeria zollingeriana, Urlica thunbergiana, Pilea spp., Boehmeria nivea and Boehmeria densiflora. The nucleotide sequences of ITS 1, 5.8S and ITS2 segments of Boehmeria frutescens are SEQ ID NO: 1, 2 and 3, respectively; the nucleotide sequences of ITS 1, 5.8S and ITS2 segments of Boehmeria zollingeriana are SEQ ID NO: 4, 5 and 6, respectively; the nucleotide sequences of ITS1, 5.8S and ITS2 segments of Urlica thunbergiana are SEQ ID NO: 7, 8 and 9, respectively; the nucleotide sequences of ITS 1, 5.8S and ITS2 segments of Pilea spp. are SEQ ID NO: 10, 11 and 12, respectively; the nucleotide sequences of ITS1, 5.8S and ITS2 segments of Boehmeria nivea are SEQ ID NO: 13, 14 and 15, respectively; and the nucleotide sequences of ITS1, 5.8S and ITS2 segments of Boehmeria densiflora are SEQ ID NO: 16, 17 and 18, respectively.

In one specific embodiment, the present invention applies the ITS sequence of Boehmeria frutescens, Boehmeria zollingeriana, Urlica thunbergiana, Pilea spp., Boehmeria nivea and Boehmeria densiflora to identify medicinal plants those are used to treat hepatitis B.

### Embodiment 1

### Extraction of the Medicinal Plant

The process for the extraction of the medicinal plant extract is as follows:

(1) Select Urticaceae plants of Boehmeria frutescens, Boehmeria zollingeriana, Urlica thunbergiana, Pilea spp., Boehmeria nivea and Boehmeria densiflora.

(2) Wash the roots or stems of the individual Urticaceae plant from step (1). Scrape the rinds and cut into flakes, and then pestle to powder.

(3) Take 600 g of powder from step (2), reflux with 3000 ml of 95% alcohol under heat for 10 hrs, filtrate the ethanol liquid extract, withdraw ethanol in the condenser, and add 1000 ml of both ethyl acetate and distilled water, and then stand for 1 hr after 30 min of stirring.

(4) Extract the water phase portion from step (3) with 500 ml of ethyl acetate and then freeze-dry to obtain the extract.

### Embodiment 2

### Bioactivity Assay of the Medicinal Plant Extract in Cell Cultured

The process for the bioactivity assay of the medicinal plant extract is as follows:

(1) Select a HBV expressing cell line HepG2.2.15 and cultured in the DMEM medium.

(2) Add 500 μg/ml of the extract from embodiment 1 to the cells of step (1) and allow 5 to cultures.

(3) Collect the cultured medium of step (2) on day 1, 3 and 5, respectively, and spin down the cells by centrifugation (2000×g for 2 min.).

(4) The supernatant from step (3) is taken out to measure the surface antigen and e antigen. The cells on plate are tested for cell cytotoxicity or HBV DNA content. After washing plate twice with buffer, 50 μl of MTT prepared in DMEM is added onto the cells and incubated at 37° C. for 30 min to 2 hrs, and then 150 μl of DMSO is added to measure the OD (optical density) at 590 nm with spectrophotometer. Cytotoxicity of the extract is based on the OD compared to the untreated group. To isolate viral DNA, wash the cells twice with buffer, add the Triton X-100 buffer and place on ice for 15 min to lyse the cells, and then collect the cell lysate into the centrifuge tube. After placing on ice for further 15 min, the cell lysate is centrifuged at 1300×g for 1 min, the supernatant and pellet are collected, respectively. Replicative intermediate DNA of HBV is isolated from the supernatant with an automatic nucleic acid extractor.

The results of bioactivty assay are described as follows:

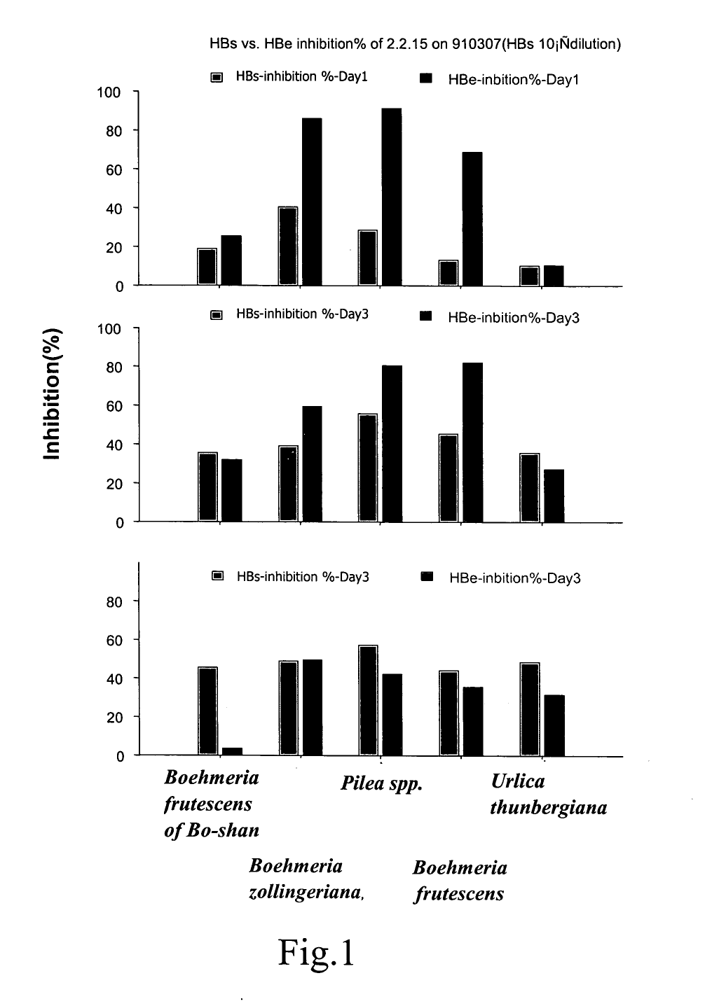

FIG. 1 is the inhibition of the Urticaceae extract against both surface antigen and e antigen of the HBV in transformed human hepatoma cells. The medicinal plant extract of Urticaceae (500 μg/ml) is added to the HepG2.2.15 cells, and the cultured medium is collected on day 1, 3 and 5, respectively. The individual HBV surface antigen and e antigen content are determined by Enzyme-Linked Immunosorbent Assay(ELISA). Our results indicated that different extracts of Urticaceae species could have diverse inhibition effects on both surface antigen and e antigen of HBV in tested cells.

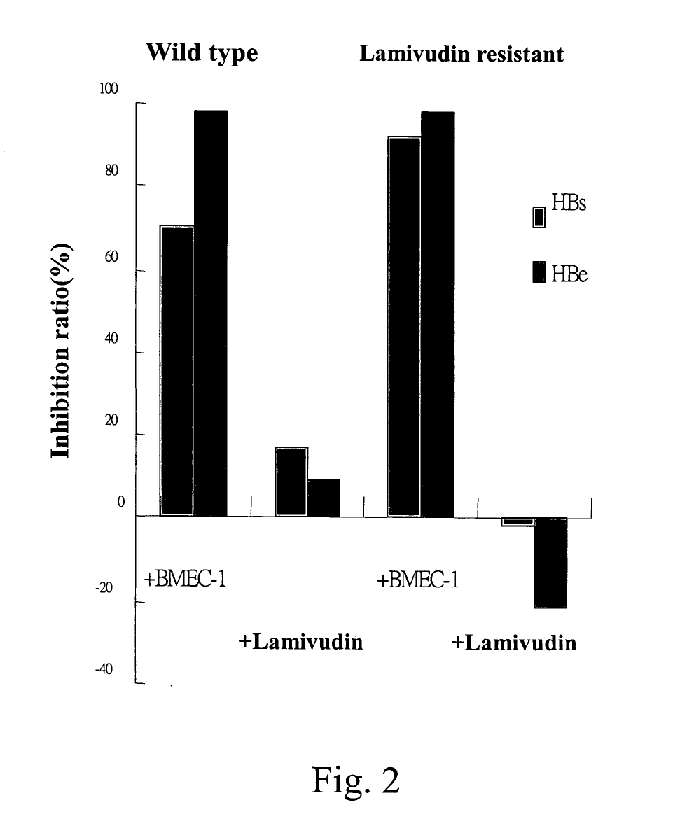

FIG. 2 is the plant extract inhibiting both surface antigen and e antigen of the wild type as well as the mutated lamivudine-resistant of HBV in transient transfected human hepatoma cells. The plasmids from the wild type and the mutated lamivudine-resistant of HBV are transfected into HepG2 and Huh7 cells, and the medicinal plant extract (500 μg/ml) or lamivudine (200 μg/ml) is added to the transfected cells, respectively. After culturing for two and four days, the media are collected, and the individual surface antigen and e antigen content of HBV is determined by ELISA. The results indicated that the plant extract could inhibit both surface antigen and e antigen of the wild type as well as the mutated lamivudine-resistant of HBV. However, lamivudine could merely inhibit the surface antigen and e antigen of the wild type but there are no effects on mutants.

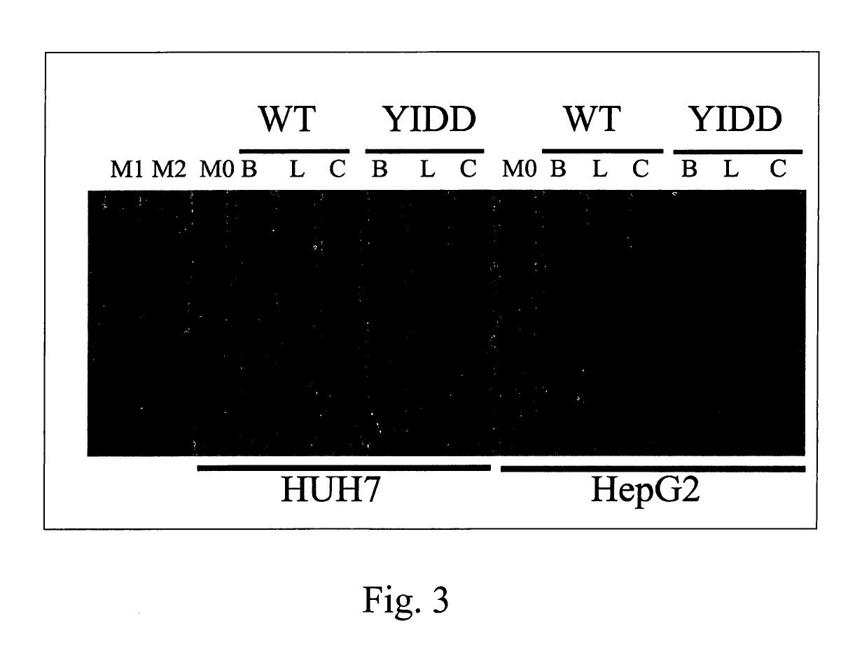

FIG. 3 is the plant extract that inhibits the viral DNA replication of the wild type and the mutated lamivudine-resistant in transfected with HBV cells. The plasmids from the wild type and the mutated lamivudine-resistant of HBV are transfected into HepG2 and Huh7 cells, respectively. The medicinal plant extract (500 μg/ml) and lamivudine (200 μg/ml) are added into the individual transfected cells, respectively. After culturing for four days, the cells are collected. After the isolation and extraction, the total DNA is separated by agarose electrophoresis and then transferred onto a specific membrane (Hybond-N). The viral DNA is hybridized with specific labeled-Dig probe and detected signal with x-ray film. The results showed that diverse inhibition effects on viral DNA replication of HBV from the different treatment in transfected cells. C is the untreated control; L is the group treated with lamivudine, which inhibits the viral DNA replication in the wild type of HBV; B is the group treated with plant extract, which shows the inhibition effect on viral DNA replication in the wild type as well as in the lamivudine-resistant of HBV.

### Embodiment 3

### Identification of Urticaceae Medicinal Plants by ITS sequence

(1) The roots and stems of Urticaceae plants are washed, the rinds are scraped to avoid microbial contamination, and cut into flakes, and then pestled to powder after freezing with liquid nitrogen.

(2) DNA extraction is first made with CTAB, PVPP and various salt concentrations, and then carried out with precipitation, centrifugation, chloroform extraction and alcohol precipitation.

(3) The primers of ITS are designed for PCR amplification, as following:

5′- CACACCGCCCGTCGCTCCTACCGA -3′
5′- ACTCGCCGTTACTAGGGGAA -3′,

Tm=60° C. for the polymerase chain reaction.

(4) The amplified DNA segments are further sequenced by the automatic nucleic acid sequencer (ABI 3100) and sequence comparison are performed by DNAMAN® software.

Wherein, the process for step (2) is as follows:
(2-1) Extraction buffer:
   100 mM Tris HCl
   20 mM EDTA
   1 M NaCl
   1% CTAB (cetyltrimethylammonium bromide)
   1% PVPP (polyvinyl polypyrrolidone, added before use)
(2-2) 3-100 mg of sample plus 0.5 ml of extraction buffer;
(2-4) incubate at 37° C. for 30 min;
(2-5) collect supernatant, and add a twofold in volume of the precipitation buffer:
   50 mM Tris HCl
   10 mM EDTA
   40 mM NaCl
   1% CTAB

20 Gently invert for 2 min;
(2-6) centrifuge at 13,000 g for 15 min and discard the supernatant;
(2-7) dissolve the precipitate from the step (2-6) in 350 μl of 1.2 M NaCl;
(2-8) RNase A treatment at 70° C. for 30 min;
(2-9) extract the sample with Chloroform: IAA=24:1, and centrifuge at 10,000 g for 5 min;
(2-10) add a 0.6-fold in volume of isopropanol, and then stand at −20° C. for 15 min;
(2-11) centrifuge at 13,000 g for 20 min at 4° C. and discard the supernatant;
(2-12) wash the precipitate with 1 ml of 70% EtOH; and
(2-13) dissolve the precipitate in TE buffer (using 10-25 μl of buffer per 20 mg of initiate).

The sequence comparison of ITS of Urticaceae medicinal plant is described as follows:

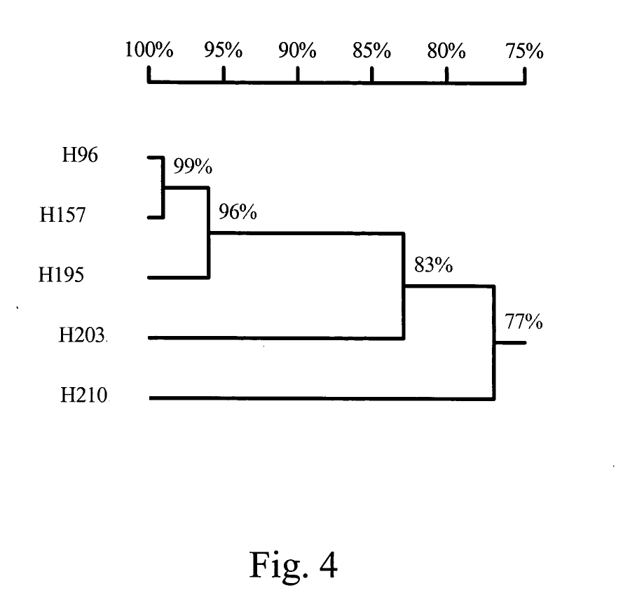

FIG. 4 is the comparison of ITS sequences resulted from above mentioned Urticaceae.

Although the present invention has been explained in relation to its preferred embodiment, it is to be understood that many other possible modifications and variations can be made without departing from the spirit and scope of the invention as hereinafter claimed.

1. A solvent extract from roots or stems of Urticaceae for treating hepatitis, said extract is extracted from an Urticaceae, and said Urticaceae has a internal transcribed spacer of rDNA in at least 70% sequence similarity compared to that of Boehmeria frutescens, Boehmeria zollingeriana, Urlica thunbergiana, Pilea spp., Boehmeria nivea or Boehmeria densiflora.

2. The solvent extract as claimed in claim 1, wherein said Boehmeria frutescens has the sequences of internal transcribed spacers from rDNA as SEQ ID NO: 1, 2 and 3.

3. The solvent extract as claimed in claim 1, wherein said Boehmeria zollingeriana has the sequences of internal transcribed spacers from rDNA as SEQ ID NO: 4, 5 and 6.

4. The solvent extract as claimed in claim 1, wherein said Urlica thunbergiana has the sequences of internal transcribed spacers from rDNA as SEQ ID NO: 7, 8 and 9.

5. The solvent extract as claimed in claim 1, wherein said Pilea spp. has the sequences of internal transcribed spacers from rDNA as SEQ ID NO: 10, 11 and 12.

6. The solvent extract as claimed in claim 1, wherein said Boehmeria nivea has the sequences of internal transcribed spacers from rDNA as SEQ ID NO: 13, 14 and 15.

7. The solvent extract as claimed in claim 1, wherein said Boehmeria densiflora has the sequences of internal transcribed spacers from rDNA as SEQ ID NO: 16, 17 and 18.

8. The solvent extract as claimed in claim 1, wherein said ITS of rDNA from one of said Boehmeria frutescens, Boehmeria zollingeriana, Urlica thunbergiana, Pilea spp., Boehmeria nivea and Boehmeria densiflora may be used for the identification of said medicinal plants for treating hepatitis B.

9. The solvent extract as claimed in claim 8, wherein said plants is identified by extracting DNA, amplifying said ITS segment of rDNA by polymerase chain reaction, sequencing said ITS segment of rDNA, and establishing the database of ITS of rDNA from Boehmeria frutescens, Boehmeria zollingeriana, Urlica thunbergiana, Pilea spp., Boehmeria nivea and Boehmeria densiflora to identify the said Urticaceae medicinal plant.

10. The solvent extract as claimed in claim 1, wherein the extraction process of said solvent extract comprises the following steps:
(a) pestling the roots or stems of said Urticaceae to obtain a powder;
(b) adding a high polar solvent or solvent mixture thereof to said powder of the step (a) to obtain a liquid extract;
and
(c) adding a solvent mixture of low polar solvent and water to the liquid phase of said liquid extract of said step (b), and then evaporating to obtain dried extract.

11. The solvent extract as claimed in claim 10, wherein said high polar solvent is methanol, ethanol or the mixture thereof.

12. The solvent extract as claimed in claim 10, wherein said low polar solvent is selected from a group comprising: ethyl acetate, difluoromethane, trifluoromethane, tetrafluoromethane, cyclohexane, n-hexane, n-butylalcohol ether, benzene, and the mixture thereof.

13. The solvent extract as claimed in claim 12, wherein said low polar solvent is ethyl acetate.

14. The solvent extract as claimed in claim 1, wherein said treating hepatitis is for hepatitis B.

15. The solvent extract as claimed in claim 1, wherein said treating further includes treating Lamivudine-resistant HBV.

16. The solvent extract as claimed in claim 1, wherein said solvent extract from the roots or stems of Urticaceae can further combinate with other drugs for treating hepatitis B.

17. A medicinal plant extract for treating hepatitis B, including treating Lamivudine-resistant HBV, and said medicinal plant extract is prepared by first pestling the roots or stems of Boehmeria frutescens, Boehmeria zollingeriana, Urlica thunbergiana, Pilea spp., Boehmeria nivea or Boehmeria densiflora, and then extracted by low polar solvent.

18. The medicinal plant extract as claimed in claim 17, wherein said medicinal plant extract is prepared by the following steps: pestling the roots or stems of the Boehmeria frutescens, Boehmeria zollingeriana, Urlica thunbergiana, Pilea spp., Boehmeria nivea or Boehmeria densiflora to obtain a powder, first extracting said powder with a high polar solvent to obtain a liquid extract, and then second extracting said liquid extract with a low polar solvent for the preparation.

19. The medicinal plant extract as claimed in claim 18, wherein said high polar solvent is selected from a group comprising: methanol, ethanol, and the mixture thereof.

20. The medicinal plant extract as claimed in claim 18, wherein said low polar solvent is selected from a group comprising: ethyl acetate, difluoromethane, trifluoromethane, tetrafluoromethane, cyclohexane, n-hexane, n-butylalcohol ether, benzene, and the mixture thereof.

21. The medicinal plant extract as claimed in claim 18, wherein said low polar solvent is ethyl acetate.

22. The medicinal plant extract as claimed in claim 18, wherein said medicinal plant extract further coordinates with other drugs for treating hepatitis.

23. A method for the identification of Urticaceae herbal medicines, comprising the following steps:
(a) providing a database of known ribosomal DNA internal transcribed spacer of an Urticaceae;
(b) providing the process of DNA extraction from roots or stems of an unidentified medicinal plant, and extracting the DNA thereof;
(c) designing a pair of primers as following, 5′-CACACCGCCCGTCGCTCCTACCGA-3′5′-ACTCGCCGTTACTAGGGGAA-3′, and amplifying the extracted DNA from said step (b) by polymerase chain reaction to obtain the ITS segment;
(d) sequencing said ITS segment of said step (c) to obtain the ITS sequence; and
(e) comparing said ITS sequence with said established database of ITS sequence, and the similarity between said ITS sequence and said ITS database for higher than 70% could be identified as an Urticaceae for treating hepatitis B of step (b).

24. The method for the identification of Urticaceae herbal medicine as claimed in claim 23, wherein said DNA extraction is performed by treating the roots or stems of said unidentified medicinal plants with extraction buffer composed of CTAB (cetyltrimethylammonium bromide), PVPP (polyvinyl polypyrrolidon) and various salt concentrations; then precipitation, centrifugation, and chloroform extraction followed by alcohol precipitation.

25. The method for the identification of Urticaceae herbal medicine as claimed in claim 23, wherein ITS sequence of said unidentified medicinal plants of the step (b) is compared to those of plants comprising: Boehmeria frutescens, Boehmeria zollingeriana, Urlica thunbergiana, Pilea spp., Boehmeria nivea and Boehmeria densiflora.
